(19) United States
(12) Patent Application Publication
(10) Pub. No.: US 2003/0108972 A1
(43) Pub. Date: Jun. 12, 2003

(54) Tethered receptor-ligand reagent and assay

(76) Inventor: Stephen Eliot Zweig, Los Gatos, CA (US)

Correspondence Address: STEPHEN E. ZWEIG, 224 VISTA DE SIERRA, LOS GATOS, CA, 95030, US

(21) Appl. No.: 10/308,411

(22) Filed: Dec. 3, 2002

Related U.S. Application Data
(60) Provisional application No. 60/339,916, filed on Dec. 6, 2001.
(60) Provisional application No. 60/389,679, filed on Jun. 17, 2002.

(51) Int. Cl. G01N 33/53

(52) U.S. Cl. 435/7.92

(57) ABSTRACT

A tethered reagent and assay is disclosed consisting of protein receptors tethered to ligands. The protein receptors can be antibodies, enzymes, hormone receptors, integral membrane proteins, and other proteins. Ligands can be antigens, enzymatic inhibitors, hormone agonists, drugs, and other protein binding ligands. The protein receptors and ligands will each be labeled with moieties capable of detecting changes in the average distance between the protein receptors and the ligand, using detection methods in which there is a sharp fall-off in signal as a function of distance. As a result, a change in the average distance between the two label moieties, such as that caused by protein-ligand binding and dissociation, produces a change in a detectable signal produced by the reagent. Tethering means may consist of flexible polymers, typically composed of a material that is chemically distinct from either the receptor or the ligand, so that the receptors and ligands may freely associate and dissociate via their specific binding sites, but not totally diffuse away from each other. When bound to solid phase surfaces, such reagents are particularly well suited for proteomic microarrays and flow cells. Such reagents may have utility for immunoassays, enzyme assays, ligand binding assays, sepsis assays, drug screening assays, and drug ADMET assays.

### BACKGROUND OF THE INVENTION

1. Field of the Invention

The invention generally concerns reagents and methods useful for enzymatic, immunochemical, and protein-ligand binding assays.

2. Description of the Related Art

This application claims the priority benefit of provisional patent applications Ser. No. 60/339,916, and 60/389,679; both entitled “Tethered receptor-ligand reagent and assay”, filed Dec. 6, 2001 and Jun. 17, 2002.

Biological systems consist of complex networks of thousands of interacting enzymes, enzyme regulators, enzyme substrates, soluble receptors, membrane receptors, biological response modifiers, and genes all acting in concert to conduct thousands of different biological processes. Although traditionally, such biological components have been studied and analyzed on a one-at-a-time basis, recent advances in biotechnology have made it clear that more global methods of study and analysis are highly desirable. For example, cancer researchers have found that cellular proliferation, cellular death, and angiogenesis, intimately involved in many aspects of cancer, are each regulated by complex networks of tens to hundreds of different cell surface receptors, enzymes and regulatory factors. A change in any one of these factors may give information relevant to the prognosis or therapy of a particular disease. Analytical methods that can globally survey these networks are thus of great medical utility for cancer research.

As a second example, consider sepsis and septic shock. As recently reviewed by Larosa (Cleveland Clinic Journal of Medicine, 69 (1), 2002, pp 65-73), sepsis and septic shock are blood stream inflammations that kill over 200,000 patients yearly in the US, cost tens of billions of dollars in medical expenses, and are the 13^{th} largest cause of death overall. The septic process begins when endotoxin, a bacterial surface molecule, binds to Toll-like receptor molecules on the lipid membranes of monocytes. This triggers the secretion of cytokines and interlukin molecules into plasma. These cytokines activate cytotoxic tissue enzymes, interfere with the normal regulation of various plasma coagulation enzymes, and additionally release tissue factor. The resulting out-of-control coagulation cascade can result in patient death in 30-50% of all septic shock patients. Because the process is both highly complex (involving both membrane bound receptors, soluble biological response modifiers, and multiple enzymes) and very fast (septic shock can often lead to a fatal outcome within hours), present day diagnostic technology is unable to accurately assess the biochemical state of a septic patient. Thus clinicians are unable to easily discern when it is appropriate to intervene with various highly expensive drugs, such as recombinant human activated protein C. Improved methods that could very rapidly track the kinetics of change in sepsis biochemical pathways would thus be of high medical interest.

As another example, modern techniques of high-throughput drug discovery generate thousands of potential drug candidates, each of which usually acts by binding to a cellular transmembrane protein, a cytoplasmic protein receptor, or an enzyme. A good drug candidate will interact only with its desired target receptor, and avoid interactions with non-target receptors. Conversely, a poor drug candidate will tend to interact non-specifically with non-target receptors.

At present, the specificity (freedom from undesired side-reactions) of such drug candidates must be evaluated by laborious, slow, and uninformative cell culture and in-vivo assays. Such assays often require significant quantities of the candidate drug, requiring that additional effort be expended in synthesizing appreciable amounts of many candidate drugs that will later be scrapped. The initial evaluation of drug candidates would be facilitated if micro-quantities of the candidate could be rapidly assayed for desired activity against a large panel of target and non-target receptors on a micro-scale. As a recent article stated:

“High-throughput screening (HTS) has proven to be a useful tool in both drug discovery and genomics research, because the ability to evaluate vast number of variables, whether they are compounds or genes, has been critical. This effort will not diminish in the foreseeable future. The cause of this trend lies in the economics, as the number of variables to be investigated increases, the cost on a per sample basis of doing so must decrease. This factor applies to both manual labor required and reagent volumes used—how to do more with less.” Astle, Drug Discovery and Development (5):5, 2002, p12.

Thus analytical methods to speed up such analysis are also of great medical utility.

As a final example, methods to rapidly screen for a wide variety of potential infectious or toxic agents are of great utility for modem diagnostics, toxicology, and defense purposes. Here, sample quantities may be limited to a small amount of material obtained from a small sample or swab of a suspected area. Improved methods to analyze small samples for a wide variety of potential toxic are thus of high utility.

The common thread between these disparate areas is the need to concentrate a large number of different biochemical detectors into a small area, and have these detectors produce useful information. Detectors of this nature are often termed microarrays. When the microarrays are used to analyze nucleic acids, they are often called “nucleic acid microarrays”. When the microarrays are used to analyze non-nucleic biochemical components, such as enzymes, protein receptors, and ligands, they are often called “proteomic microarrays”. Proteomic microarrays in turn may be designated as “immunochemical-microarrays” if they work via antibody binding reagents, “enzymatic-microarrays” if they work via enzyme and enzyme substrate reagents, and “membrane microarrays” if they work with membrane components.

Microarray technology: In recent years, microarrays have become widely used for genomic and proteomic biotechnology, biomedical research, and biomedical diagnosis. In particular, microarray methods have become widely used for nucleic acid research, and a large number of nucleic acid microarrays are commercially available from Affymetrix Inc., Incyte Pharmaceuticals, Inc., and many other companies. These methods (reviewed in Schena, Microarray Biochip Technology (2000) Eaton Publishing , Natick, Mass.) generally work by binding a large number of nucleic acid microsamples to the surface of a flat support. Samples containing one or more unknown complementary nucleic acids are then exposed to the nucleic acid microarray, and the sample is allowed to hybridize to the microarray. Hybridized nucleic acids are then detected by various means, and the overall nucleic acid composition of the unknown sample is assessed.

The general principle has been that to detect two biologically interacting elements that form a pair, such as complementary nucleic acid strands, the microarray will contain one-half of the pair, the unknown analyte will contain the other half of the pair, and the interaction between the two elements will generate a detectable signal.

A good overview of protein microarray technology in general, as it exists at the time of this patent application, can be found in the article by Mitchell, “A perspective on protein microarrays”, Nature biotechnology (20), 2002, 225-229, the contents of which are incorporated herein by reference.

In contrast to the intense research and commercial activity currently ongoing with nucleic acid microarrays, and the equally intense modern interest in proteomics, there is comparatively little research or commercial activity with enzymatic or immunochemical microarrays. This is because these types. of microarrays pose a number of major technical challenges. For nucleic acid microarrays, nucleic acid hybridization methods make the reactions easy to detect. Here, hybridization of an analyte's single stranded nucleic acid to a complementary single stranded nucleic acid microarray element always generates a double stranded nucleic acid structure. This double stranded structure can be easily detected regardless of nucleic acid sequence.

By contrast, enzyme, protein receptor, and immunochemical microarrays have been considerably more difficult to implement. This is due to the more unique and individual nature of each different enzymatic and immunochemical reaction. In contrast to the generic double stranded nucleic acid structure generated by nucleic acid microarrays, each different element of an enzymatic, protein receptor, immunochemical or membrane microarray may potentially require its own unique detection chemistry. This is because for enzymes and receptors, each enzyme, ligand, and substrate has its own unique chemical structure. For antibodies, although the antibody part of an immunochemical microarray is comparatively more standardized, each antigen has its own unique chemical structure.

Consider enzyme-substrate microarrays: in order to detect enzymatic reactions in the microarray format, prior art teaches that either the enzyme or the substrate must be bound to the solid microarray surface. If the analyte consists of a solution of unknown enzyme substrates or enzyme inhibitors, then an array of enzymes must be bound to the microarray surface. If the analyte consists of a solution of unknown enzymes, then a complex array of enzyme substrates must be bound to the microarray surface. Each situation generates its own unique set of reaction detection problems.

The case where the test analyte consists of a solution of unknown enzyme substrates, enzyme inhibitors, or response modifiers poses the most challenging detection problems, and here the microarray prior art is largely silent.

The case where the analyte consists of a solution of unknown enzymes is somewhat more tractable. Here, prior art teaches that analyte solutions consisting of unknown protein kinases may be analyzed by binding protein kinase substrate peptides to the surface of a microarray, allowing the array to react with the protein kinase containing analyte, and a source of radioactive phosphorus, which is usually p³² or p³³ labeled adenosine triphosphate (ATP). The microarray is then washed and those peptides that were phosphorylated are detected by autoradiography. These methods were taught by MacBeath and Schreiber, Science 2000, 289, 1760-1763; and Falsey Renil et al, Bioconjugate Chem. 2001, 12, 346-353.

Although demonstrations have shown that such methods can work for simple test cases, these methods are not widely used in practice. Radioactivity detection methods suffer from the general safety concerns attending the use of radioactive materials, and the slow speed of autoradiography methods. Another drawback is the difficulty in obtaining reaction kinetic data, since only one time points worth of data can be obtained from any single microarray.

At present, immunochemical microarrays also exist more as technical “proof of principle” demonstrations, rather than as practical devices. Prior art teaches that antibodies may be bound to a microarray surface. These antibody-microarrays have been used in “proof of principle” demonstrations to analyze mixtures of demonstration “unknown” antigens where the “unknown” antigens have been previously labeled with a detectable moiety, such as fluorescent tag. Although useful for demonstration purposes, real biological samples do not have antigens that are conveniently labeled with such tags. Thus such methods have limited utility.

The case where the microarray consists of an array of bound antigens, and is used to detect unknown antibodies, is particularly useful for allergists and specialists in autoimmune disorders. This case is most easily addressed by prior art. Here each antibody that binds to an antigen on a microarray is likely to be from the same species (e.g. all human antibodies, etc.). Additionally, antibodies are large molecules that themselves present multiple epitopes to other antibodies from a different species. Thus, for example, a microarray consisting of various bound antigens may be reacted with a solution of unknown human antibodies, and assessed by washing the microarray, and then reacting the microarray with a detection reagent composed of fluorescently labeled anti-human antibodies.

Use of peptide microarrays, constructed by photochemical methods, for antibody recognition of peptide patterns was taught by Fodor et. al., Science 1991, 251, 767-773 and U.S. Pat. No. 5,143,854. Here, peptides were synthesized on solid phase supports by photochemical means, and probed with mouse monoclonal antibodies against the peptide, and fluorescently labeled goat-anti-mouse monoclonal antibodies.

Use of peptide microarrays for antibody binding or protein-protein binding was taught by MacBeath and Schreiber, Science 2000, 289, 1760-1763. Here glass slide microarrays were chemically activated to covalently bind peptides, and various peptides and proteins were spotted onto the slides using conventional spotting equipment. The peptides formed a covalent bond with the derivitized glass. They spotted unlabeled protein-G onto the slides, and demonstrated that fluorescently labeled immunoglobulin G (IgG) could bind to the protein-G portions of the glass slide microarrays. They also spotted antibodies against steroid digoxigenin (DIG) onto other microarrays, and demonstrated that these bound antibodies could bind DIG that had been coupled to fluorescently labeled bovine serum albumin. Techniques of this type were also taught by Falsey, Renil et al, Bioconjugate Chem. 2001, 12, 346-353.

### RESONANCE ENERGY TRANSFER TECHNOLOGY

Resonance energy transfer detection techniques are widely used in immunochemical assays, and other ligand binding assays.

Resonance energy transfer (RET) occurs when a photon (light) emitting first moiety comes close (usually 7-9 nanometers or less) to a second moiety capable of absorbing the photons emitted by the light emitting first moiety. Here the photon directly transfers from the first moiety to the second moiety. As a result, the light emitted by the first moiety can be quenched by the second moiety. Alternatively, the wavelength of the light emitted by the first moiety can wavelength shifted and re-emitted by the second moiety. Either event causes a detectable change in the signal emitted by the first light-producing moiety. Typically, when the two moieties are separated by a distance greater than about 7-9 nanometers, resonance energy transfer no longer occurs. Thus RET methods can be used to deduce the average distance between the two moieties.

The light-emitting moiety may be stimulated to emit light through a number of different mechanisms. The two mechanisms most commonly used are fluorescence and luminescence.

Fluorescence-quenching methods (alternatively often called Fluorescence Resonance Energy Transfer or “FRET” methods) have been previously used for numerous types of binding assays. Such methods generally rely upon placing a first fluorescent label moiety on one side of the binding pair, and a second fluorescence quencher or fluorescence wavelength shifter moiety on the other side of the binding pair, whereby fluorescence is extinguished or wavelength shifted upon binding. Using these methods, the presence of unlabeled “test” antigen in an unknown analyte sample can be detected by the displacement of a labeled reagent-antigen from a labeled reagent antibody. These methods were taught by U.S. Pat. Nos. 4,174,384; 4,261,968; and others.

U.S. Pat. 5,229,302 teaches a fluorescence immunoassay using a pseudo-antigen coupled to a fluorescence quencher and a fluorescence labeled antibody, wherein an analyte containing antigen may displace the quencher labeled pseudo-antigen and restore antibody fluorescence.

These techniques were also taught by Lee et. al., “Fluorescent excitation transfer immunoassay for the determination of spinosyn A in water”: J Agric Food Chem (1999) Jul;47(7):2766-70. Lee et. al. developed a fluorescence excitation transfer immunoassay using fluorescence labeled antigen and a tetramethylrhodamine labeled antibody. The nonlabeled antigen competed for the labeled antigen, and reversed fluorescence quenching.

Aurora BioSciences Corporation, San Diego, Calif. produces the PhosphoryLIGHT™ kinase/phosphatase system for determining the phosphorylation state of a peptide using FRET techniques. Here fluorescein and coumarin are used as the fluorescent moiety-quencher pair.

U.S. Pat. No. 5,981,200 teaches a method for enzymatic analysis using a fluorophore and a quencher linked by a peptide.

U.S. Pat. No. 6,197,928 teaches a fluorescent indicator protein containing an analyte binding region, a protein linker, and a protein ligand binding region labeled with a second peptide group that acts as a fluorescence quencher. This indicator is a single protein, capable of being synthesized by living cells (either by transduction with the nucleic acid sequence for the protein, or by microinjection). The fluorescent and quencher moieties are fluorescent and light absorbing proteins such as Aequorea, green fluorescent protein, and the like. Binding of the ligand moiety to the quencher moiety caused a conformational rearrangement of the protein resulting in a change in fluorescence resonance transfer efficiency when the fluorescent protein moiety was excited by irradiation.

U.S. Pat. Nos. 5,723,591 and 5,925,517 describe various types of self-quenching fluorescence nucleic acid probes.

RET techniques have been used for monitoring glucose. U.S. Pat. No. 6,163,714 teaches glucose detection techniques relying upon changes in RET between labeled concanavalin-A and labeled dextran. Here, the comparatively large concanavalin-A and dextran molecules were entrapped in microparticles by a membrane that was impermeable to large molecules, but permeable to small molecules such as glucose. This membrane prevented the two-labeled components from diffusing far away from each other, but allowed glucose to enter.

Although such impermeable membrane methods are useful for low molecular weight analytes, such as glucose, which can diffuse through small pore membranes, they are clearly unsuitable for higher molecular weight analytes, such as proteins, that would be blocked by the same membrane barrier used to prevent the labeled receptor and labeled ligand from diffusing away.

U.S. Pat. No. 6,379,622 teaches methods of monitoring glucose concentrations by utilizing the changes in the RET signal induced by the differential binding of labeled concanavalin-A to glucose, and quantum dot reference methods for signal normalization.

Luminescent RET methods are discussed by Selvin and Hearst, “Luminescence energy transfer using a terbium chelate: improvements on fluorescence energy transfer”; Proc. Natl. Acad. Sci. 91 (1994): 10024-10028; and Nakagawa et. al. Anal Biochem 2001 Feb 1;289(1):77-81.

### IMMUNOCHEMICAL TECHNOLOGY

Immunochemical assays are widely used for medical diagnostics and biomedical research. Many immunochemical assays work by binding either the antigen or the antibody to the solid support, and incorporating one or more label moieties onto the antigen, antibody, or both so that binding may be assessed.

U.S. Pat. No. 4,977,077 teaches an integrated solid-phase immunoassay in which an antibody and a fluorescently labeled ligand are complexed to a solid phase, and an unlabeled ligand displaces the labeled ligand into the liquid phase, where it is measured.

U.S. Pat. No. 5,599,681 teaches antibodies that specifically bind to the phosphorylated or dephosphorylated state of a protein.

### TETHERING METHODS

Miyata et. al., Nature 399 (1999) 766-769 disclose a rversibly antigen-responsive hydrogel formed by binding antibody and antigen to an acrylamide polymer network. Conjugating antibodies to dendrimers was taught by Barth et. al., Bioconjug Chem January-February, 1994, 5(1):58-66 U.S. Pat. No. 6,083,708 teaches methods to couple two different polypeptides to dendrimers forming dendrimer-polypeptide complexes. U.S. Pat. No. 6,121,056 teaches dendrimer immunoassay methods where antibody mixes with multiple specificities are bound to solid phase surfaces divided into multiple sub-areas by interstices on the solid phase surface. U.S. Pat. No. 5,861,319 teaches immobilization of specific binding assay reagents to solid phase surfaces.

U.S. Pat. No. 5,998,588 teaches interactive molecular conjugates.

### SUMMARY OF THE INVENTION

Here, an improved reagent, useful for the creation of improved proteomic microarrays, analyte detecting flow cells, cytological stains/probes, and other analytical devices, is taught. This reagent is useful for studying the effect that unknown “test ligands” have upon the ability of a receptor of interest (here called the “target receptor”) to bind to a reagent ligand that is present as part of the assay reagent (here called the “reagent ligand”). The design of the improved reagent departs from that of conventional proteomic microarray reagents, which generally bind only target receptor reagents (antibodies, enzymes, or ligand receptors) to the surface of the microarray, or which generally bind only reagent ligand reagents (antigen, haptein, enzyme substrate, receptor ligand) to the surface of the microarray. Here, the improved reagent binds both the target receptor and it's corresponding reagent ligand to the surface of the analytical device, in a loosely tethered form.

In the present invention, the target receptor and reagent ligand are free to associate and dissociate, while remaining connected to the device. These association-dissociation reactions are detected by detection techniques, such as resonance energy transfer, where the efficiency of energy transfer varies sharply as a function of distance between a first excitation source and a second energy emission source. Generally one of the target receptor-reagent ligand pair members is associated with an excitation source, and the other member of the pair is associated with an emission source. Binding of unknown test ligands to the target receptor perturbs the interaction between the target receptor and the reagent ligand, changing the amount of energy transfer between the two. This results in a change in the detectable signal emitted by the reagent.

The improved reagent of this invention typically contains:

(1) A target receptor labeled with a first energy emitting moiety;

(2) A reagent ligand capable of binding to the target receptor, and labeled with a second moiety capable of exchanging energy (usually by resonance transfer) with the first moiety, such that the reagent produces a detectable signal that changes when the reagent ligand binds or dissociates from the target receptor.

(3) A flexible tether or tethers, different from either the target receptor or the reagent ligand, that binds both the target receptor and reagent ligand into a single linked structure, while preserving the ability of the reagent ligand to associate and dissociate from its binding site on the target receptor. This tether allows the reagent ligand to diffuse a sufficient distance away from the target receptor that this diffusion can be detected by the sharp change in energy transfer efficiency as a function of distance.

(4) An optional anchor means to anchor the reagent to a surface of interest.

The first moiety will typically generate a detectable signal that is a fluorescent or luminescent signal. The second moiety will typically be a fluorescence or luminescence quencher or wavelength shifter that RET alters the signal of the first moiety when the second moiety is close to the first moiety. This RET distance is typically 10 nanometers or less.

The flexible tether will typically be a natural or synthetic polymer, usually hydrophilic in nature. The reagent's target receptor and reagent ligand are tightly coupled to the tether so as to have negligible dissociation from the tether under normal reaction conditions. Such coupling means may be by covalent bond, or high affinity non-covalent bond. The tethering means are chosen so that the reagent's target receptor and reagent ligand have sufficient steric flexibility to be able to further bind to each other via the target receptor's reagent ligand-binding site. The tether should be long enough as to enable the target receptor and reagent ligand, when not otherwise bound at their mutual binding sites, to diffuse far enough away as to significantly diminish RET.

Binding of the tethered reagent ligand to the tethered target receptor will normally result in a high level of RET, usually producing wavelength shifting or quenching of the detectable signal. Addition of test ligand will displace the labeled reagent ligand from the labeled target receptor, and diminish the quenching or wavelength shifting of the detectable signal.

The binding of the tethered reagent ligand to its tethered target receptor may be modulated by other means as well. The tethered reagent ligand may be modified by enzymatic modification (phosphorylation, dephosphorylation, glycosylation, deglycosylation, proteolysis, etc.) or other means so that the modified reagent ligand has either a greatly enhanced or greatly diminished affinity for its target receptor. Alternatively, the tethered target receptor may be modified by enzymatic modification, the action of a biological response modifying agent upon an allosteric site of the tethered target receptor, or other means so that the modified target receptor has either a greatly enhanced or greatly diminished affinity for its tethered reagent ligand. Any of these cases may be detected by a RET induced change in the detectable signal emitted by the first moiety. For brevity, these various effects will often be designated as a “factor” that modulates the target receptor binding to its reagent ligand.

The tethered receptor ligand reagent may be used directly in liquid phase assays. Alternatively, in a more favored embodiment, the tethered receptor-ligand reagent is tightly bound to a support surface.

The reagent, when bound to a support, exhibits a number of distinct advantages over prior art reagents. All of the chemistry required to perform the desired analysis is present on the support, and no additional reagents are required. Thus the support may be stored as a unit, exposed to a test ligand, and the kinetics of test ligand action upon the reagent observed without the necessity of applying additional reagents or performing additional operations. Because the reagent contains all necessary detection means, the test ligand itself does not need to be artificially labeled in any way. Moreover, upon the completion of the test, the test ligands may be washed away, and the reagent reused or regenerated for subsequent assays. The end result is a simpler and more versatile assay.

This support may be a simple support or “test strip” that contains large amounts of only a few types of reagents. In this situation, the reagent may be applied to the support by simple dipping, coating, spraying, or other means. Alternatively, the support may be a microarray capable of supporting hundreds or thousands of different reagents of the present invention. In this situation, the reagent will be applied by common microarray sample spotting methods, such as slotted pens, jet printing, and the like. In other situations, the support may be a flow cell component, such as the inside of a capillary tube, or the surface of an optical fiber, in which case more specialized fabrication methods may be required.

Microarrays incorporating reagents of the present invention may be used for a number of different purposes. The microarrays may be immunochemical microarrays, used to analyze complex mixtures of antigens. The microarrays may be enzymatic microarrays, used to analyze complex mixtures of enzymes for patterns of enzymatic activity. The microarrays may be biological response modifier characterization microarrays, used to analyze drugs, cellular extracts, biological extracts, toxins, etc. for specific activity against hundreds or thousands of different protein receptors or enzymes.

In an alternate embodiment, the reagents of the present invention may be incorporated into flow-cell devices and used to characterize candidate drugs for desired patterns of binding to target and non-target receptors. Here, the micro scale and reusable properties of the reagent, coupled with the small volumes of materials required for capillary and other micro scale flow-cells, enable comprehensive tests to be performed using only micro-quantities of test material.

In some cases, it may be preferable to prepare each tethered receptor-ligand reagent as a completely separate synthesis, and bind these separately synthesized tethered reagents to a relatively simple “passive” support surface. In other cases, it may be preferable to employ an “active” support surface with a number of the “generic” components of the tethered receptor-ligand reagent (for example some of the tethering and/or detection components) pre-prepared on the support surface. Here, a user may create the final microarray by simply spotting the “raw” receptor-ligand components (e.g. the untethered target receptors and reagent ligands) onto the active surface. The spotted target receptors and reagent ligands can then bind to the tethering groups (and possibly also the detection groups) on the active surface without any further user effort. This later option reduces the amount of labor required to produce a large number of different tethered-ligand detector elements.

### BRIEF DESCRIPTION OF THE DRAWING

FIG. 1 shows a diagram of a tethered antibody-ligand reagent detecting free antigens

FIG. 2 shows multiple tethered antibody-ligand reagents bound to a solid phase support.

FIG. 3 shows a diagram of a tethered antibody-ligand reagent detecting an enzymatically-modified reagent ligand

FIG. 4 shows a diagram of a tethered receptor-ligand reagent detecting a modified target receptor.

FIG. 5 shows a diagram of general-purpose microarray surface in which the target receptor and reagent ligand are tethered to an active surface.

FIG. 6 shows a diagram of a tethered antibody-ligand reagent that additionally contains both a tethered enzyme, and a tethered surface attachment group.

FIG. 7 shows a diagram of a tethered receptor-ligand reagent binding to a transmembrane protein.

FIG. 8 shows a schematic diagram of the pulsed-light epifluorescent microscope apparatus used to read the tethered-ligand reagents.

FIG. 9 shows a flow cell incorporating reagents of the present invention.

### DETAILED DESCRIPTION OF THE INVENTION

Detection Methods:

Detection methods suitable for the present invention include luminescence resonance energy transfer (luminescence, bioluminescence, chemoluminescence, electrochemoluminescence, etc.) methods, fluorescence resonance energy transfer methods, and other methods that produce a detectable signal that is altered by close proximity between two moieties. Other detection methods include evanescent wave techniques, electrochemiluminescence, electron transport, surface plasmon resonance, and other techniques in which excitation energy varies as a sharp gradient away from an excitation surface.

Luminescence energy transfer methods are particularly useful. As discussed by Selvin and Hearst “Luminescence energy transfer using a terbium chelate: improvements on fluorescence energy transfer”; Proc. Natl. Acad. Sci. 91 (1994): 10024-10028; fluorescence energy transfer methods suffer from a number of drawbacks. The distance that energy transfer can occur (typically 1-7 nanometers) is a bit short to study many receptor ligand-binding situations. The signal to noise levels can suffer from to cross talk between due to “interfering fluorescence from the fluorophore and direct excitation of the receptor”. An additional problem is that the efficiency of energy transfer is a function of both the distance and the relative angle between the energy emitter moiety and the energy acceptor moiety. Thus although fluorescence energy transfer methods are widely used, these methods are not necessarily optimal for high performance microarrays where large numbers of different receptors and ligands need to be linked by a standardized chemistry, and where good signal-to-noise performance is imperative.

By contrast, luminescence energy transfer methods have a number of distinct advantages. They have a superior signal-to-noise ratio because cross-talk due to inadvertent direct excitation of the receptor moiety by the external excitation energy does not occur. The energy transfer can operate over longer distances (up to 9 nm or longer), and the efficiency of energy transfer is much less dependent on the relative angle between the energy emitter moiety and the energy transfer moiety.

Suitable luminescence resonance energy transfer moieties include the chelates or conjugates of the luminescent lanthanide elements terbium and europium, with organic dyes such as CY-5 (discussed by Selvin and Hearst, previously cited). Nakagawa et. al. Anal Biochem Feb. 1, 2001;289 (1): 77-81 report a 10-fold improvement sensitivity over previous fluorescence energy transfer methods when the bioluminescent protein moieties luciferase and yellow fluorescent protein are used in immunoassays. Other examples include Ruthenium (Ru) metal-ligand complex luminescent moieties paired with Nile-blue or TOTO-3 dyes; etc. Use of smaller synthetic energy transfer moieties over larger biological protein energy transfer moieties may be favored in situations where the larger size of the biological protein may distort protein-receptor-ligand binding due to steric effects.

Luminescence may be induced by various means, including light excitation, chemical excitation, electrochemical excitation, etc. In the case where luminescence is induced by light excitation, and the luminescent reagents are incorporated into a microarray, it may be advantageous to observe the microarray using a pulsed light source, and electronic means wherein the luminescent excited RET signal of the microarray elements is observed at one or more intervals after the light excitation pulse.

In the case where fluorescence resonance energy transfer methods are desired, suitable labels include fluorescein labels with tetramethylrhodamine (TMR) RET transducers per the methods of Lee et. al., J Agric Food Chem July, 1999, 47(7):2766-70, and others.

Tethering Means:

The tethering means should have a number of characteristics. The tether should tightly bind both the target receptors and the reagent ligands. It should allow them enough steric freedom of movement to effectively both associate and dissociate. It should also have a loose enough structure so that an unbound test ligand or factor can displace the labeled reagent ligand from the target receptor. Polymers suitable for tethering include hydrogels such as the acrylamide and N,N′-methylenebisacrylamide crosslinked gels of Miyata et. al., (A reversibly antigen-responsive hydrogel, Nature 399 (1999), 766-799). Other suitable polymers include dextran, polyethylene glycol, hydroxymethylacrylate, peptides, dendrimers, and the like.

Peptides may also be used as tethering polymers for the present invention. In the unlinked state, to prevent RET, the tether should enable the receptor and ligand to spend about 90% or more of the time separated by a distance of 7-9 nm or greater. Thus peptide sequences that form extended filamentous, rather than globular, conformations are preferred. Peptides composed of 1-amino acids tend to be susceptible to proteolysis. To reduce proteolysis, use of tethers composed of d-amino acids may also be preferred, as amino acids are typically more resistant to proteolysis.

Due to the tethering means, the reagent ligand will remain in close proximity to the target receptor even when the reagent ligand is not directly bound to the receptor site itself. Thus, everything else being equal, a tethered reagent ligand will have a binding advantage over a test ligand. In order to enable the tethered reagent ligand to be successfully displaced by a test ligand, in some cases, it may be advantageous to use a synthetic reagent ligand with somewhat lower affinity for the target receptor than the test ligand.

Linking Receptors and Ligands to Tethers:

Protein receptors and ligands may be linked to acrylamide polymers by chemical modification following the methods of Miyata et. al., Nature 399 (1999) 766-769. For example, antibody target receptors and protein reagent ligands may be are reacted with either N-acryloyloxysuccinimide or N-succinimidyl acrylate (Polysciences Inc, Warrington, Pa.) by incubation in 0.2M phosphate buffer solution at pH 7.4 for one hour to produce vinyl-IgG, or vinyl-ligand. These derivitized antibodies and ligands may, in turn, be derivitized with acrylamide, forming long tethered polymers (hydrogels) using the redox initiators Ammonium persulfate/tetramethylenediamine (TEMED). This technique is described in more detail by Adalsteinsson et. al., Preparation and magnetic filtration of polyacrylamide gels containing covalently immobilized proteins and a ferrofluid, J. Mol. Catalysis (6), 1979, 199-225, the contents of which are incorporated herein by reference.

Microarray Binding Methods

The reagents of the present invention may be bound to microarray surfaces by either their protein moieties or their linker moieties. Binding by the protein moieties can be done using the techniques of MacBeath and Schreiber. Science 2000, 289, 1760-1763; Falsey. Renil et al. Bioconjugate Chem. 2001, 12, 346-353, or other methods. Typically superaldehyde conjugated glass microarray slides are obtained from TeleChem International Inc., Arryit division, (Sunnyvale, Calif.): These slides contain aldehyde groups covalently bound to the glass surface. When incubated with lysine containing proteins, such as bovine serum albumin (BSA), the aldehydes react with the primary amines on the protein forming a Schiff's base linkage. This covalently binds the protein to the glass slide.

Because, in many instances, direct binding of the protein moiety to the microarray surface may result in distortion or inactivation of the protein, it will often be advantageous to bind the reagent of the present invention to microarray surfaces using the reagent's tether moiety. In this way, the protein moieties can minimize distortion while remaining fully attached to the microarray. In this latter case, suitable attachment methods will be largely dependent upon the choice of tether in question. Often, a convenient way to do this will be to incorporate a “surface anchor” antibody (such as an anti-BSA antibody) to the tether reagent, along with the other tethered test components. This tethered “anchor” antibody will bind the tethered reagents to antigens (such as bound BSA) bound on the surface of interest.

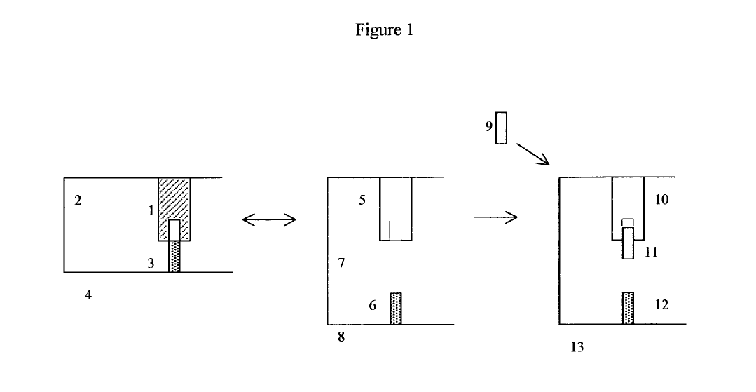

FIG. 1 shows an example of a tethered receptor-ligand reagent in which the target receptor is an antibody, the reagent ligand is an antigen (or haptein), and the reagent is used in an immunoassay to detect test antigens. (The situation where the protein receptor is not an antibody, but rather is a different type of protein able to bind ligands is essentially the same). In this example, the reagent consists of a target antibody (1) coupled to a detectable signal emitting first moiety (not shown). The antibody is tightly coupled to a flexible supporting polymer (2) by either a covalent link, or by high binding strength non-covalent link groups (such as an avidin-biotin link). The reagent further contains a reagent ligand (3) (which can be a haptein or antigen) capable of binding to the binding site of target antibody (1). Reagent ligand (3) contains a second moiety that can exchange energy by resonance transfer with the first label moiety (not shown). The binding affinity between the target antibody (1) and reagent ligand (3) is sufficiently high that the vast majority of the reagent consists of a form in which the target antibody and reagent ligand are bound, and the energy emitted by the first moiety on the target antibody is altered by the close proximity of the second moiety on the reagent ligand by RET. This is shown as (4). Typically, the affinity of this target antibody-reagent ligand pair is chosen so that the target antibody will have a higher affinity towards a test antigen, and will preferentially bind to a test antigen if it is present.

Although the affinity of the antibody-ligand reagents are chosen so as to have the vast majority of the reagent present in the bound form where the detectable signal emitted by the first moiety is altered by the second moiety, the affinity of the target antibody-reagent ligand pair is also selected as to not be so high that the pair never dissociates. Occasionally, due to random thermodynamic fluctuations, the target antibody-reagent ligand bond dissociates, even although the target antibody (5) and reagent ligand (6) remain tightly associated to the flexible supporting tether (7). This is shown as (8). When this occurs, the signal emitted by the first moiety on antibody (5) is no longer altered by the second moiety on ligand (6), resulting in a detectable signal that is not altered by RET. Typically this will be a luminescent or fluorescent signal at a defined set of wavelengths. This will create a faint unaltered signal background.

When this reagent is exposed to the test antigen (9) that the target antibody (10) has affinity against, the antibody will preferentially bind to the target antigen (11) over the labeled reagent ligand (12). This will stabilize the non RET form of the reagent (13), leading to an increase in the non RET form of the detectable signal when the test antigen is present.

Note that no separation step is required, and that this will work when the tethered reagent is both free in solution, and when the tethered reagent is bound to a solid phase surface.

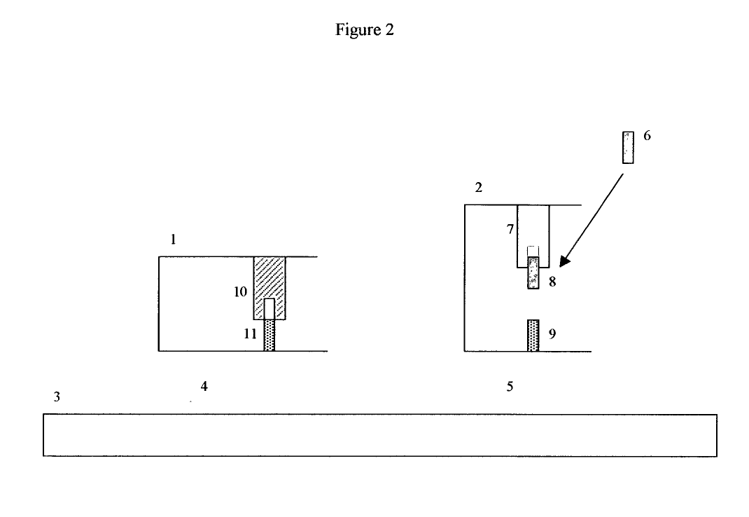

FIG. 2 shows an example of a microarray immunoassay using multiple tethered antibody-ligand reagents (from FIG. 1) bound to a solid surface. In this example, two different reagents (1), (2) specific for two different antigens are bound to different locations on solid support (3) through linking groups (4, 5). Also in this example, the first label moiety on each target antibody is a fluorescent moiety, and the second label moiety on each antigenic reagent ligand (haptein) quenches the fluorescence of the first moiety.

In this example, the microarray is exposed to an analyte containing test antigen (6) reactive to fluorescent-labeled target antibody (7). Test antigen (6) binds to target antibody (7) forming complex (8) that displaces the tethered reagent ligand (9) labeled with a fluorescence quencher (not shown). As a result, the fluorescence of reagent (2) is unquenched, and this zone of the microarray exhibits enhanced fluorescence.

By contrast, in this example, the analyte does not contain an antigen reactive to fluorescent-labeled target antibody (10) on reagent (1). As a result, target antibody (10) remains bound to its fluorescent quencher labeled tethered reagent ligand (11). As a result, this zone of the microarray exhibits reduced fluorescence.

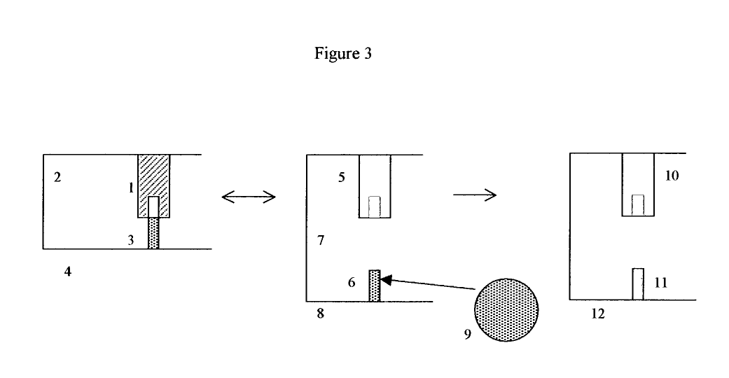

FIG. 3 shows an enzyme assay using tethered reagents. As before, the reagent consists of a target antibody (1) reactive with a first enzymatically modifiable state of a reagent ligand, (3) but not reactive with a second enzymatically modifiable state of the reagent ligand (3). This target antibody (1) is coupled to a detectable signal emitting first moiety (not shown). The antibody is also tightly coupled to a flexible supporting polymer (2) by either a covalent link, or by high binding strength non-covalent link groups (such as an avidin-biotin link).

The reagent's enzymatically modifiable reagent ligand (3) (a.k.a. haptein, antigen) further contains a second moiety that can exchange energy by RET with the first label moiety (not shown). The binding affinity between the target antibody (1) and enzymatically modifiable reagent ligand (3) is sufficiently high that the vast majority of the reagent consists of a form in which the target antibody and reagent ligand are bound. Here, the first signal moiety on the target antibody is altered by the close proximity of the second moiety on the reagent ligand. This is shown as (4).

The affinity of the target antibody-reagent ligand pair is chosen so that when the reagent ligand is present in a first enzymatically modifiable state, the vast majority of the reagent is present in the bound form, where the detectable signal is altered by RET. Note the affinity of the target antibody-reagent ligand pair is selected as to not be so high that the pair never dissociates. Rather, due to random thermodynamic fluctuations, the antibody-ligand bond may allow occasional dissociation. (Note that antibody (5) and reagent ligand (6) remain tightly associated to the flexible supporting polymer (7)). This is shown as (8). When this occurs, the second moiety on reagent ligand (6) no longer alters the detectable signal emitted by the first moiety on antibody (5). This will create a faint unaltered signal background, even when all of the reagent ligand is present in a first enzymatically modifiable state.

When this tethered reagent is exposed to a test enzyme (9) that enzymatically modifies reagent ligand (6) from a first state to a second state (e.g. phosphorylation, dephosphorylation, glycosylation, deglycosylation, etc.) the antibody (1) will no longer have affinity for the reagent ligand (11). This will stabilize the non RET form of the reagent (12), leading to an increase in the non RET signal when the test enzyme is present. Alternatively, the antibody may be selected to prefer the enzymatically-modified form of the reagent ligand. In this case, enzymatic activity will result in an increase in the RET form of the signal.

In addition to immunochemical techniques, where the tethered target receptor is an antibody, alternative tethered target receptor types may be used to analyze other types of analytes. For example, the tethered target receptor may be a tethered enzyme or a tethered receptor to a biological response modifier. In this case, the reagent ligand may be an enzyme substrate, enzyme inhibitor, biological response modifier molecule (hormone, etc.) or an agonist to a biological response modifier. Alternatively, test enzyme (9) may alter the state of target receptor (5), rather than the reagent ligand (6). In either case, enzymatic activity is detected.

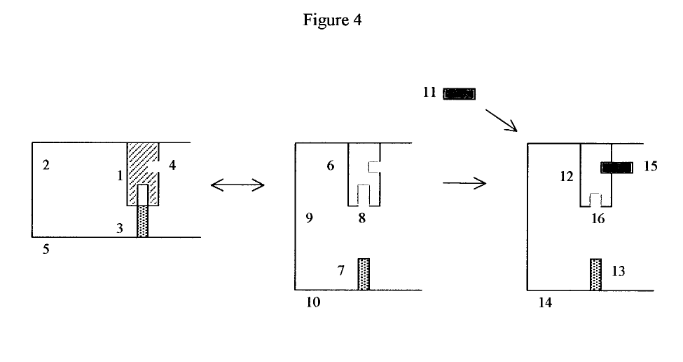

FIG. 4 shows an example where the tethered receptor-ligand assays are used to screen for test factors that modify the ligand binding activity of a target receptor. In this application, a target receptor, labeled with a first moiety that emits a detectable signal (1), is bound to the flexible tether (2). The tethered reagent further contains a reagent ligand (3) capable of binding to the target receptor. Ligand (3) is labeled with a second moiety that can exchange energy by RET with the first moiety (not shown). Target receptor (1) may also contain an optional site (4) (for example, an allosteric site) where an external factor may act to modify the ligand binding ability of the receptor.

The binding affinity between the target receptor (1) and reagent ligand (3) is sufficiently high that the vast majority of the reagent consists of a form in which the receptor and reagent ligand are bound, and the detectable signal emitted by the first moiety on the target receptor is altered by the close proximity of the second moiety on the reagent ligand. This is shown as (5).

Although the affinity of the receptor-ligand reagents are chosen so as to have the vast majority of the reagent present in the bound form (5) for at least one receptor state, the affinity of the receptor-reagent ligand pair is usually not so high that the pair never dissociates. Occasionally, due to random thermodynamic fluctuations, the receptor-ligand bond dissociates from the receptor ligand-binding site (8), even although the receptor (6) and reagent ligand (7) remain tightly associated to the flexible supporting polymer (9). When this occurs, the detectable signal emitted by the first moiety on target receptor (6) is no longer altered by the second moiety on reagent ligand (7), resulting in a faint unaltered signal background. This is shown as (10).

When this tethered reagent is exposed to an external test factor (11) that can modify the state of target receptor (12), so that receptor (12) has altered affinity for reagent ligand (13), the ratio between the two states (5, 10) of the reagent will be altered, resulting in a change the detectable signal emitted by the reagent. In this example, the external factor is assumed to have stabilized the unbound form of the reagent. This is shown as (14).

Test factor (11) can act upon target receptor (12) in a number of different ways and by a number of different mechanisms. For example, test factor (11) could be an enzyme that modifies the state of target receptor (12) (e.g. phosphorylation, dephosphorylation, proteolytic cleavage, glycosylation, etc.) as to alter the binding affinity between target receptor (12) and the reagent ligand (13). Alternatively, test factor (11) could bind to an allosteric site (4, 15) on target receptor (12) and alter its affinity for reagent ligand (13). Finally, test factor (11) could bind to the reagent ligand-binding site (16) on target receptor (12) and directly block the binding of reagent ligand (13) to target receptor (16).

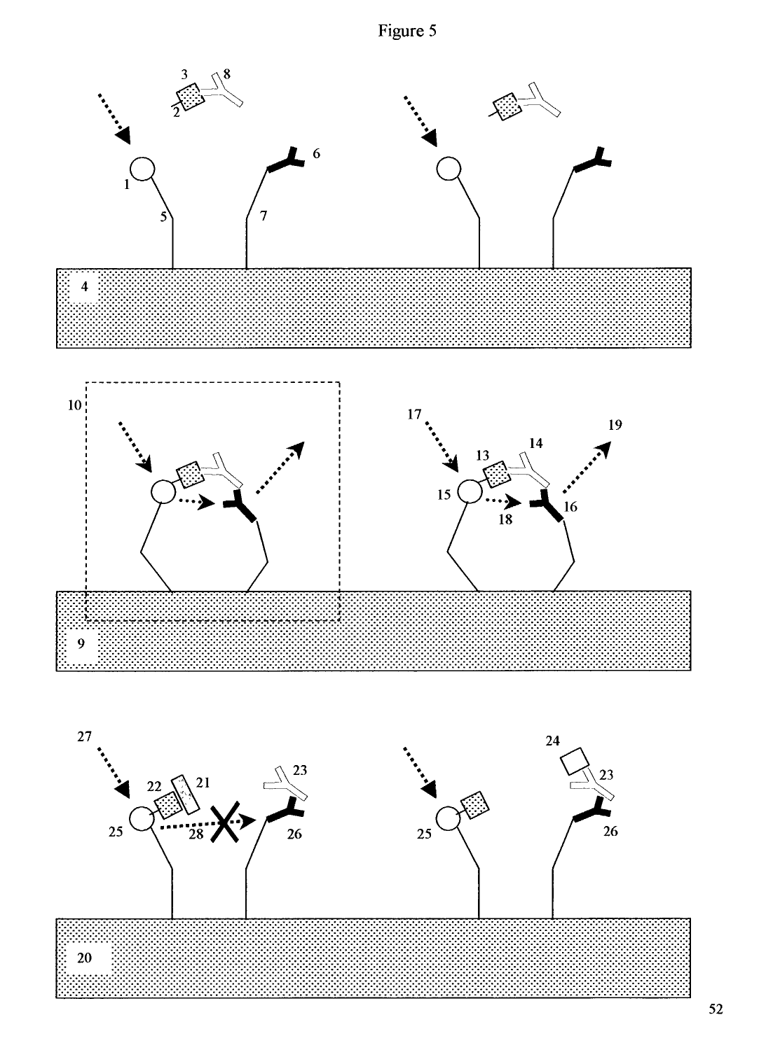

The process of producing a large number of custom tethered receptor-ligand reagents requires a large number of coupling reactions, which is laborious. In many cases, it will be advantageous to produce a more generic detector surface containing many of the elements of the tethered receptor-ligand assay in a pre-prepared form, so that simple application of the “raw” receptors and reagent ligands to the pre-prepared detector surface in one or more locations will create the complete tethered receptor-ligand reagent. An example of such a pre-prepared “active” detector surface is shown in FIG. 5.

FIG. 5 shows the details of a general-purpose “active” microarray surface constructed using general-purpose tethered receptor-ligand reagents. Here a first active surface receptor (1), such as avidin or strepavidin, which is capable of binding a wide variety of different labeled (2) reagent ligands (3) is bound to surface (4) by a first linker group (5). A second active surface receptor (6), is bound to solid surface (4) by a second linker group (7). Second active surface receptor (6) will typically bind to a target receptor (8), and this target receptor (8) will typically bind to reagent ligand (3) at least some of the time. Often, target receptor (8) will be an antibody, such as a mouse monoclonal antibody against reagent ligand (3), and second active site receptor (6) will often be an antibody (such as an anti-mouse antibody) against target receptor (8).

Active surface receptors (1) and (6) will typically be intermixed on active surface (4) with tether groups (5) and (7) long enough so that active surface receptors (1) and (6) are able to come into close contact with each other. Active surface receptors (1) and (6) will usually be selected as to have no binding affinity for either each other, or for tether groups (5), (7). Active surface receptors (1) and (6) will usually be labeled with moieties capable of exchanging resonance transfer energy exchange with each other when active surface receptors (1) and (6) are in close association.

An array of different detector zones may be created upon active surface (4) by the addition of one or more additional target receptors and reagent ligands to different regions of the active surface (4). Surface (9) shows an example of the resulting bound complex (10).

In a bound complex such as (10), a reagent ligand such as a biotinated reagent ligand (13) bound to a target receptor (14) specific for target receptor (13) (for example, a mouse monoclonal antibody against (13)) is applied to the active surface (9). A sandwich structure is formed, in which a first active surface receptor (15) binds to biotinated biotinated reagent ligand (13), a second active surface tethered receptor (16) binds to the target receptor (14), and the target receptor (14) continues to bind to biotinated reagent ligand (13). As a result, labeled first and second active surface receptors (15) and (16) are brought into close proximity. As a result, energy (17) may be applied to a first moiety on first active surface receptor (15), transferred (18) to a second moiety on second active surface receptor (16) by resonance transfer effects, and be re-emitted (19) by the second moiety on second active surface receptor (16).

Surface (20) shows a detail of the mechanism by which bound complex (10) acts to detect molecular interactions induced by external test ligands or factors. In this example, a test ligand or factor (21) or (24) changes the binding between the reagent ligand (22) and its target receptor (23). Example of test factors can include test ligands (21) that bind to reagent ligand (22) and alter the binding between reagent ligand (22) and its target receptor (23); test agents (24) that bind to target receptor (23); and test agents that modify either the state or conformation of either reagent ligand (22) or target receptor (23) so as to alter the binding between reagent ligand (22) and target receptor (23). In this later case, such test agents can act by enzymatic modification of reagent ligand (22) or target receptor (23), by binding to an allosteric receptor on reagent ligand (22) or target receptor (23), or by other means. When this binding is altered so as to cause reagent ligand (22) and target receptor (23) to dissociate, the distance between first active surface receptor (25) and second active surface receptor (26) increases. As a result, resonance energy transfer between moieties on first active surface receptor (25) and second active surface receptor (26) is reduced. As a result, application of energy (27) to first active surface receptor (25) results in diminished transfer of energy (28) to second active surface receptor (26). A smaller signal is thus generated by second active surface receptor (26).

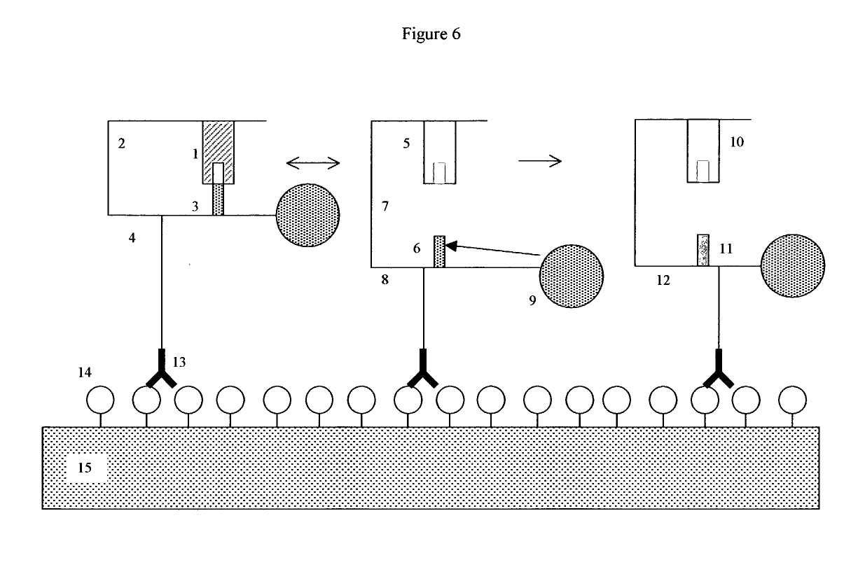

FIG. 6 shows an alternate form of an enzyme inhibitor assay using tethered reagents. Here the enzyme itself is also bound to the tether (either directly, or through an antibody-tether link that is not shown). The tethered reagents are in turn bound to a BSA coated microarray surface via a tethered anti-BSA antibody.

As in FIG. 3, the reagent consists of a target antibody (1) reactive with a first enzymatically modifiable state of a reagent ligand, (3) but not reactive with a second enzymatically modifiable state of the reagent ligand (3). This target antibody (1) is coupled to a detectable signal emitting first moiety (not shown). The antibody is also tightly coupled to a flexible supporting polymer tether (2) by either a covalent link, or by high binding strength non-covalent link groups (such as an avidin-biotin link).

The reagent's enzymatically modifiable reagent ligand (3) (a.k.a. haptein, antigen) further contains a second moiety that can exchange energy by RET with the first label moiety (not shown). The binding affinity between the target antibody (1) and enzymatically modifiable reagent ligand (3) is sufficiently high that the vast majority of the reagent consists of a form in which the target antibody and reagent ligand are bound. Here, the first signal moiety on the target antibody is altered by the close proximity of the second moiety on the reagent ligand. This is shown as (4).

The affinity of the target antibody-reagent ligand pair is chosen so that when the reagent ligand is present in a first enzymatically modifiable state, the vast majority of the reagent is present in the bound form, where the detectable signal is altered by RET. Note the affinity of the target antibody-reagent ligand pair is selected as to not be so high that the pair never dissociates. Rather, due to random thermodynamic fluctuations, the antibody-ligand bond may allow occasional dissociation. (Note that antibody (5) and reagent ligand (6) remain tightly associated to the flexible supporting polymer tether (7)). This is shown as (8). When this occurs, the second moiety on reagent ligand (6) no longer alters the detectable signal emitted by the first moiety on antibody (5). This will create a faint unaltered signal background, even when all of the reagent ligand is present in a first enzymatically modifiable state.

This tethered reagent also contains a tethered test enzyme (9) that enzymatically modifies reagent ligand (6) from a first state to a second state (e.g. phosphorylation, dephosphorylation, glycosylation, deglycosylation, etc.) when the enzyme is exposed to the appropriate cofactors (such as ATP for a tethered kinase enzyme, etc.) When the tethered enzyme (9) is activated by exposure to the appropriate cofactors, the antibody (1) will no longer have affinity for the reagent ligand (11). This will stabilize the non RET form of the reagent (12), leading to an increase in the non RET signal when the test enzyme is present. Alternatively, the antibody may be selected to prefer the enzymatically-modified form of the reagent ligand. In this case, enzymatic activity will result in an increase in the RET form of the signal.

However if an enzyme inhibitor, such as a drug candidate test ligand, is present in the reaction buffer, the activity of test enzyme (9) will be inhibited. Thus, the enzymatic conversion of the reagent ligand will not occur, and this inhibition process will be detected by the RET signal emitted by the reagent.

In this figure, the tethered reagent (4, 8, 12) shown additionally contains a surface attachment group (13), such as an anti-BSA antibody, that itself is attached to the polymeric tether (2). This surface attachment group (13) binds to anchor groups (14), such as BSA, which in turn are attached to surface (15).

Note that more than one type of tethered enzyme (9) may be attached. In many cases, it may be advantageous to bind a first enzyme that performs a given chemical modification along with a second enzyme that reverses the chemical modification. For example, a drug discovery kinase-phosphatase detection element can be constructed by tethering a mixture of kinase and phosphatase enzymes to tether (2). Drugs that inhibit either kinase or phosphatase activity will tend to drive the reagent into either the all-unbound form (8) or all bound form (4). Since the reagent is attached to a surface (15), which may be a flow cell surface, multiple drugs may be assessed by using the same tethered reagents, followed by a washing cycle between each new test drug.

Although surface (15) may be the surface of a flow cell or microarray, other configurations are also possible. In an alternative embodiment, surface 15 may be a biological structure of interest, such as a cell component, and surface attachment group (13) may be an antibody against an epitope (14) present on the biological structure of interest. In this alternative embodiment, the tethered reagent may be used as a cytological probe that binds to a cellular region of interest, and reports on the enzymatic or other factor activity present in the environment near the region of interest.

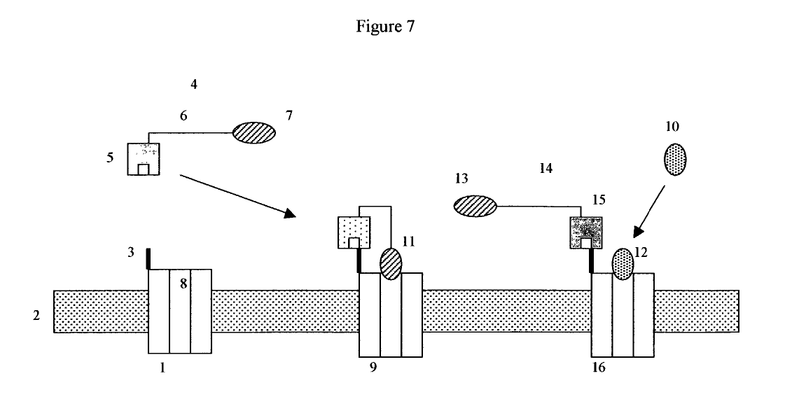

FIG. 7 shows an alternate soluble tethered receptor ligand reagent that is particularly useful for membrane protein receptors (and enzymes), which have limited hydrophilic surface areas available for labeling. Here target membrane protein (1) in lipid bilayer membrane (2) is labeled with a single binding moiety (3), such as a biotin label. This alternative soluble tethered receptor-ligand reagent (4) consists of a reagent receptor, such as avidin, (5) capable of binding-to-binding moiety (3). Reagent receptor (5) contains a tether (6) that tethers reagent receptor (5) to a reagent ligand (7) capable of binding to a ligand binding receptor (8) on target membrane protein (1). Reagent receptor (5) is additionally labeled with a first label moiety that emits a detectable signal, and reagent ligand (7) is additionally labeled with a second transducer moiety that can exchange energy by RET with the first label moiety on reagent receptor (5).

The tethered reagent (4) binds to binding moiety (3) on target membrane protein (1) forming a bound complex (9). In the absence of free test ligand (10) that is capable of binding to receptor (8) on target membrane protein (1), the tethered reagent ligand (7) is capable of binding to ligand receptor (8) on target membrane protein (1). This bound form of the tethered ligand is shown as (11). When this happens, the RET label moiety on reagent ligand (7) is brought close to the signal moiety on reagent receptor (5), resulting in a change in the detectable signal.

In the presence of test ligand (10), the test ligand binds to the ligand binding receptor (8) on target membrane protein (1). This is shown as (12). Bound test ligand (12) will prevent the tethered reagent ligand (7,13) on tethered reagent (4,14) from binding to ligand binding receptor (8). This is shown as (16). As a result, the RET label moiety on reagent ligand (13) is further away from the signal moiety on reagent receptor (5), and does not modify the detectable signal.

Note that in this example, reagent receptor (5) does not bind tethered reagent ligand (7) directly. Rather, reagent receptor (5) binds tethered reagent ligand (7) indirectly through target membrane protein (1).

Certain variations on this scheme are also possible. For example, tethered reagent ligand (7) may contain the detectable signal emitting moiety, and reagent receptor (5) may contain the RET transducer moiety. Alternatively, binding moiety (3) can do double duty as a signal-emitting moiety. As another alternative, target membrane protein (1) can be labeled with both a binding moiety (3) and a signal-emitting moiety (such as green fluorescent protein, aquelorin, etc.). Finally, binding moiety (3) may be an epitope (or haptein) on target membrane protein (1), and reagent receptor (5) may be an antibody against epitope (3).

The soluble alternate form of the tethered reagent shown in FIG. 7 may have particular use for drug Absorption, Distribution, Metabolism, Excretion, and Toxicity (ADMET) studies. Here, previous work (summarized in Darvas and Dorman, High-Throughput ADMETox Estimation: In Vitro & In Silico Approaches, (2002) BioTechniques Press, Eaton Publishing, Westborough, Mass.) has shown that analysis of a drug's ability to interact with the cytochrome P450 family of membrane proteins (responsible for drug metabolism, excretion, and toxicity) and the ABC cassette transport membrane proteins (such as p-glycoprotein, responsible for drug distribution and excretion) can give much valuable ADMET information. Here, useful in-vitro ADMET assays may be constructed by binding membrane preparations containing the purified cytochrome P450 and/or ABC cassette transport proteins to the surface of flow cells, and in-turn binding tethered reagents where the reagent ligand portion of the reagent ligand is an inhibitor of the ADMET membrane protein of interest. Candidate drugs that displace the tethered reagent ligands are introduced to the flow cell. Those candidate drugs that bind to the appropriate membrane targets then can be quickly identified by changes in the RET signal.

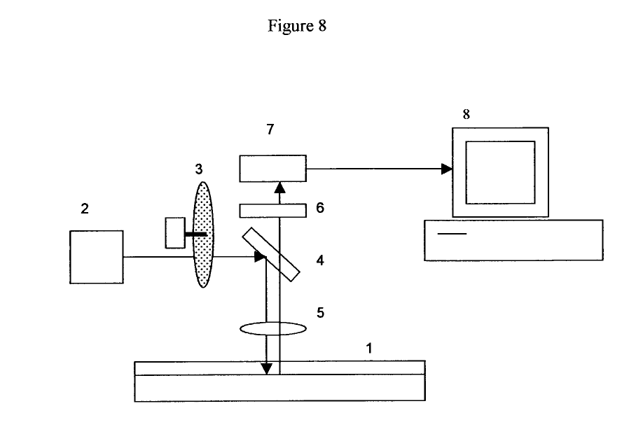

FIG. 8 shows a diagram of the pulsed-light epifluorescent microscope experimental apparatus used to obtain data from microarrays incorporating the tethered-ligand reagent. Here the membrane microarray (1) is first illuminated by a light source (2) that passes through a rotating wheel light-chopper (3) to obtain brief pulses of illumination. This illumination passes through a bandpass mirror (4). This illumination is focused by objective (5) onto the microarray. The return fluorescence signal passes back through objective (5) though bandpass filter (6) and into photomultiplier (7). The signal is then digitized and analyzed by computer (8).

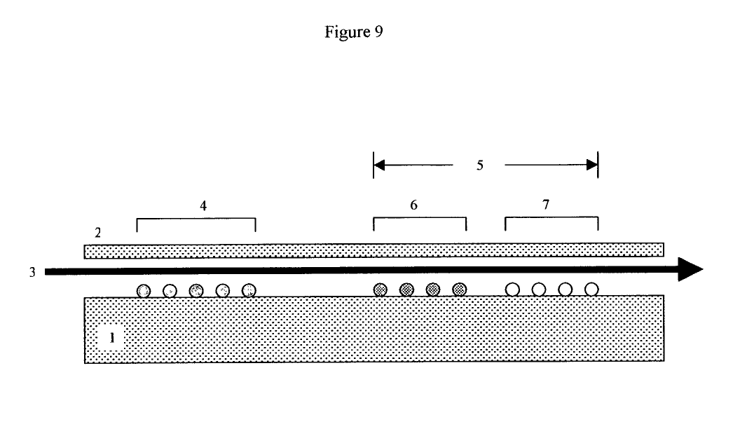

FIG. 9 shows a reusable flow cell containing a tethered receptor-ligand microarray (1), and a transparent cover (2). Samples, such as various drug discovery candidate test ligands, wash buffers, control samples, etc. flow through the cell on a sequential basis (3). Here, the flow cell contains both a drug screening section (4) containing samples of different target receptor enzymes, and an ADMET section (5) that gives immediate feedback as to the potential suitability of a given candidate test ligand for drug use. Here, the ADMET section contains a portion with various cytochrome P450 drug detoxification enzymes (such as CYP3A4) (6), and a portion with various ABC drug transporter proteins (such as P-glycoprotein) (7). Other ADMET detectors may also be added as appropriate.

### EXAMPLE 1

Active Surface Preparation

Avidin is labeled with a luminescent lanthanide conjugate, and rabbit anti-mouse IgG antibody is fluorescently labeled with a the long wavelength fluorescent dye CY-5, following the methods of Selvin and Hearst (Luminescence energy transfer using a terbium chelate: improvements on fluorescence energy transfer PNAS 91 (1994): 10024-10028). The reaction mix also includes an unlabeled goat anti bovine serum albumin (BSA) antibody. These reaction components are conjugated by reaction with N-acryloxysuccinimide, (Polysciences Inc. Warrington, Pa.), to produce free vinyl groups, and then co-polymerized with free acrylamide monomer, cross linking agent (N, N-methylenebisacrylamide) ammonium persulfate, and TEMED to form a loose hydrogel with protein groups tethered together by acrylamide polymers, following the methods of Miyata et al., (Nature 399 (1999) 766-769), Shoemaker et. al. (Shoemaker S., Hoffman A., Priest J., Synthesis and Properties of Vinyl Monomer/Enzyme Conjugates, Applied Biochemistry and Biotechnology 15 (1987), p 11-24) and Adalsteinsson et. al. (Preparation and magnetic filtration of polyacrylamide gels containing covalently immobilized proteins and a ferrofluid, J. Mol. Catalysis 6 (1979) 199-225). The resulting hydrogel contains luminescent avidin, fluorescent rabbit anti mouse antibody, and goat anti BSA antibody all tethered together on long acrylamide polymers. This hydrogel may then be processed by mechanical sheering and dilution to the proper consistency for coating.

BSA is covalently bound to treated glass slides following the techniques of Macbeath (MacBeath G, Schreiber S “Printing proteins as microarrays for high-throughput function determination” Science, 2000, 289(5485):1760-3). As described previously, superaldehyde conjugated glass microarray slides are obtained from TeleChem, International Inc., Arryit division, (Sunnyvale, Calif.). These slides contain aldehyde groups covalently bound to the glass surface. When incubated with lysine containing proteins, such as bovine serum albumin (BSA), the aldehydes react with the primary amines on the protein forming a Schiff's base linkage. This covalently binds the protein to the glass slide.

The dilute hydrogel solution is coated on the BSA-coupled glass slides, and is anchored to the glass slides via the anti-BSA antibody on the hydrogel anchoring to the glass slide bound BSA (some details of this type of antibody mediated anchoring mechanism are shown in FIG. 6 (13), (14), and (15)). Unbound hydrogel is removed by washing. The active slides may then be treated with an optional preservative solution (such as trehalose or glycerol), dried, and stored until use.

### EXAMPLE 2

a Procalcitonin Reactive Microarray Detection Element

As previously discussed, sepsis is a complex biochemical cascade of different inflammatory events. To perform with high detection efficiency, a useful sepsis diagnostic should be able to simultaneously track a variety of different levels of enzymes and biological factors, including coagulation factors such as activated protein c, various cytokines, various interlukins, and other factors.

As discussed by U.S. Pat. No. 6,077,665, procalcitonin levels are among the more useful sepsis markers. It is likely that any future multi-component sepsis diagnostic will contain a procalcitonin sensitive detection element. This example shows how such a procalcitonin sensitive microarray detection element may be created.

A procalcitonin reactive mouse monoclonal antibody, such as PCT-N1 may be obtained from Biovendor laboratory medicine (612 00 Brno Czech Republic) or alternative vendor. Biotinated whole procalcitonin, or a biotinated procalcitonin peptide, such as Val-Gly-Ala-Pro-Gly-Lys-Lys-Arg-Asp-Met-Ser-Ser (taught by U.S. Pat. No. 6,541,311) may be obtained from the American Peptide Company, Sunnyvale Calif. (Often, however, it may be preferable to use a protease resistant synthetic procalcitonin peptide analog, made from d-amino acids or other protease resistant subunits, to minimize the effects of contaminating proteases in the test sample.)

To construct the procalcitonin reactive microarray detection element, a mixture of biotinated-procalcitonin and PCT-N1 is prepared in a buffer containing 40% glycerol. This mixture is spotted onto one or more zones on the previously prepared active surface, and allowed to react. The biotinated-procalcitonin peptide attaches to the luminescent tethered avidin groups, while the mouse PCT-N1 antibody attaches to the fluorescent tethered rabbit anti-mouse antibody.

After the reaction, the microarray is washed to remove unbound material, and again stored with an optional preservative solution until used.

To perform the assay, the microarray is exposed to a plasma sample from a patient suspected of having sepsis. The microarray is observed by using an epifluorescence microscope using a pulse (chopped) light source for excitation, and a bandpass filter/photomultiplier based detection system. The luminescent lanthanide groups attached to the avidin molecule are excited during the “on” pulse of illumination. After the “on” pulse is complete, the luminescent lanthanide groups in turn emit luminescent light, which is transferred to the fluorescent Cy-5 dye on the rabbit anti-mouse antibody by resonance transfer. A bandpass filter is employed on the photomultiplier detection system to enable the detector to only detect the wavelengths emitted by the fluorescent Cy-5 dye. As a result, when rabbit anti-mouse bound PCT-N1 antibody binds to the avidin bound procalcitonin peptide, the Cy-5 group on the rabbit anti-mouse antibody is brought close to the luminescent avidin molecule, producing a relatively high level of RET. Thus the Cy-5 group will emit a relatively strong signal.

However if the patient test sample contains a high level of procalcitonin, the free test procalcitonin competes with the tethered procalcitonin antibody for PCT-N1 binding. The mouse monoclonal PCT-N1 antibody will tend to not bind to the tethered peptide, and thus the relative distance between the luminescent avidin group and the Cy-5 labeled rabbit anti-mouse antibody will increase. Thus the Cy-5 group will emit a relatively weak signal. The higher the levels of procalcitonin in the test sample, the weaker the Cy-5 signal.

Using these principles, immunochemically based microarrays containing large number of detection elements to the many elements of the sepsis cascade (such as protein c, d-dimer, endotoxin, lipoproteins, coagulation factors, etc) may be created, and incorporated into a single device that is capable of a rapid and comprehensive analysis of a septic patient using only trace amounts of patient plasma.

### EXAMPLE 2

A Protein Kinase Detecting Flow-Cell.

Microarrays containing specific enzymatic activity detection zones may also be constructed by spotting the appropriate reagents onto discrete portions of an active surface. In this example, a kinase-phosphatase sensitive microarray zone may be created by using a mouse monoclonal antibody against a specific phosphorylation state of a kinase target peptide as the target receptor, and a biotinated kinase target peptide as the reagent ligand.

For this demonstration kinase-phosphatase assay, biotinated-ENDYINASL (Biotin-Glu-Asn-Asp-Tyr-Ile-Asn-Ala-Ser-Leu-OH) may be obtained from AnaSpec, Inc., San Jose, Calif. and used as an example target peptide. ENDYINASL is a general peptide substrate for protein tyrosine phosphatases (Daum et. al., Anal Biochem 1993 May 15;211(1):504). Alternatively, as desired, more specific target peptides may be used.

Again for demonstration purposes, a wide specificity mouse monoclonal antibody against phosphotyrosine, such as MAB3109, may be obtained from Chemichon International, Temecula, Calif. This antibody binds with high affinity to the phosphorylated tyrosine, but not to unphosphorylated tyrosine. Alternatively, more sequence specific antibodies may also be used.

To construct the kinase-phosphatase reactive microarray zone, a mixture of biotinated-ENDYINASL and MAB3109 is prepared in a buffer containing 40% glycerol. This mixture is spotted onto one or more zones on the previously prepared active surface, and allowed to react. The biotinated-EDNYINASL peptide attaches to the luminescent tethered avidin groups, while the mouse MAB3 109 antibody attaches to the fluorescent tethered rabbit anti-mouse antibody.

After the reaction, the microarray is washed to remove unbound material, and again stored with an optional preservative solution until used.

To perform the assay, the microarray is generally incorporated into a small flow cell, and observed by using an epifluorescence microscope using a pulse (chopped) light source for excitation, and a bandpass filter/photomultiplier based detection system. The luminescent lanthanide groups attached to the avidin molecule are excited during the “on” pulse of illumination. After the “on” pulse is complete, the luminescent lanthanide groups in turn emit luminescent light, which is transferred to the fluorescent Cy-5 dye on the rabbit anti-mouse antibody by resonance transfer. A filter is employed on the photomultiplier detection system to enable the detector to detect the longer wavelengths emitted by the fluorescent Cy-5 dye. As a result, when rabbit anti-mouse bound MAB3190 binds to the phosphorylated form of the avidin bound ENDYINASL peptide, the Cy-5 group on the rabbit anti-mouse antibody is brought close to the luminescent avidin molecule, producing a relatively high level of RET. Thus the Cy-5 group will emit a relatively strong signal. Conversely, when the ENDYINASL peptide is not phosphorylated, the mouse monoclonal antibody will not bind, and thus the relative distance between the luminescent avidin group and the Cy-5 labeled rabbit anti-mouse antibody will increase. Thus the Cy-5 group will emit a relatively weak signal.

To characterize the response of the microarray kinase-phosphatase detection zone, the flow cell will be alternatively flushed with appropriate control kinase and phosphatase enzymes, as well as essential cofactors such as ATP and magnesium. A phosphatase enzyme flush will dephosphorylate all the tyrosines on the target ENDYINASYL peptide, producing the lowest (low control) level of RET signal. Conversely, a Kinase+ATP flush will phosphorylate all of the tyrosines on the target EDNYINASYL peptide, producing the highest (high control) level of RET signal. This serves to characterize the relative sensitivity of each microarray detection zone. This calibration technique is discussed in more detail in Example 3 below. After this initial characterization, the microarray peptides may be restored to their desired state (either all phosphorylated or all dephosphorylated) by exposure to the appropriate conditioning kinase or phosphatases. Excess enzyme will then be washed out of the reaction cell, and the response of the reaction zone to unknown test analytes may then be assessed.

### EXAMPLE 3

Signal Processing:

Signal Processing: For this type of test, the useful information for any given test condition is the percent of the reagent ligands that are binding to the tethered receptors. To obtain this data, calibration and signal processing of the microarray data will usually be required. This is because any given microarray spot will have both variations in the concentration of tethered reagents per spot, as well as a variable background signal.

In this example, it is assumed that the tethered receptors have moieties that emit photons. These photons are absorbed by a second moiety on the tethered reagent ligand by RET effects, and then remitted at a shifted (longer) wavelength. Thus then when all the tethered reagent ligands are bound, the second moiety will emit a high intensity of the shifted wavelength signal. Conversely, when all the tethered receptors are unbound, the second moiety will emit a low intensity of the shifted wavelength signal.

Here, R_{b} represents the fraction of tethered reagent ligand in a bound form, and R_{f} represents the fraction of tethered reagent ligand in a free form. Thus in the case where 100% of the tethered reagent ligand is bound to the receptor, R_{b}=1, and R_{f}=0. Conversely, in the case where 0% of the tethered reagent ligand is bound to the receptor, R_{b}=0, and R_{f}=1.

Each tethered receptor will generate a shifted wavelength signal, W, which is proportional to the relative ratio of the free (unbound) receptors (R_{f}) to the reagent-ligand bound receptors (R_{b}).

Thus W=m(R_{b}/(R_{f}+R_{b})) where m is an efficiency constant.

In fractional terms: R_{f}+R_{b}=1 so therefore on a per tethered receptor basis:

W=m(1-R_{f}).

So the total signal “S” from any given microarray spot will be proportional to the output per tethered receptor, “W” times the number of tethered receptors, “x” so that:

S=m(1-R_{f})x+b; where b is a background signal.

To determine the percentage of free receptors, R_{f}for any given microarray spot, the efficiency constant “m”, the number of tethered receptors “x”, and the background signal “b” must be determined by a calibration process.

One good way to obtain this calibration data is to incorporate the reagent spots into a flow cell. Here, each reagent spot may be calibrated by first exposing the reagent to a low-level control solution containing no test ligands (or a control solution that forces the receptors to totally bind to the reagent ligands), and determining a first control signal Sᵢ. The flow cell is then exposed to a high-level control solution containing a saturating concentration of test ligands (or a control solution that forces the receptors to totally dissociate from the reagent ligands), and a second control signal S₂ is obtained.

Since for the zero test ligand control case, S₁, all the tethered receptors are bound to the reagent ligands; so R_{f}=0, and R_{b}=1. Thus:

S₁=m(1-0)x+b; or alternatively S₁=mx+b

By contrast, for the high test ligand control case S₂, all the tethered receptors are free to diffuse away from the reagent ligands, so R_{f}=1 and R_{b}=0. Thus:

S₂=m(1-1)+b; or alternatively S₂=b

Thus S1-S2=mx;

To determine the percentage of free (R_{f}) or bound (R_{b}) tethered receptors for intermediate levels of unknown test ligands producing a signal Sₜₑₛₜ, the Sₜₑₛₜ signal is processed using the data obtained from the S₁and S₂control data by

Sₜₑₛₜ=m(1-R_{f})x+b

and since S₁-S₂=mx; and S₂=b, then

Sₜₑₛₜ=(S₁-S₂)(1-R_{f})+S₂

Solving for the percentage of free receptors, R_{f}, for any given value of Sₜₑₛₜgives:

(Sₜₑₛₜ-S₂)/(S₁-S₂)=(1-R_{f})

giving

R_{f}=1-(Sₜₑₛₜ-S₂)/(S₁-S₂)(3)

After the flow cell is calibrated with the low and high control solutions, and the S₁ and S₂ (3) values are recorded for each reagent spot, the flow cell is then regenerated by flushing out the high control test ligands with excess buffer. After the regeneration cycle, the experimental test ligands are then added to the system, and the “Sₜₑₛₜ” experimental signal levels processed using the previously obtained S₁and S₂control values as in equation (3) above.

### EXAMPLE 4

Kinase Inhibitor Drug Discovery Assay

The protein kinase/phosphatase flow cell from example 2 may also serve as the basis of a drug discovery assay. This example shows an assay to detect candidate drugs that inhibit tyrosine kinase.

This reagent may be prepared in one of several ways. In one of the simplest ways, a mixture of biotinated kinase peptide substrate, mouse monoclonal antiphosphotyrosine antibody, a mouse monoclonal antibody against a target kinase of interest, and the target kinase of interest itself are applied to the active surface from example 2. Using this mixture, the kinase of interest will become attached to the same tether as the peptide substrate and antiphosphotyrosine antibody by virtue of the mouse antikinase monoclonal antibody-tethered rabbit anti-mouse antibody link. (Alternate ways to couple the kinase enzyme of interest to the tether such as directly conjugating the kinase enzyme with vinyl groups and including it in the acrylamide copolymerization mix, may also be used.) This active surface is then incorporated into a flow cell, which is calibrated by the methods described in examples 2 and 3. The overall configuration is similar (but not identical) to FIG. 6.

In use, the flow cell is first run with a buffer containing soluble phosphatase enzymes and no ATP. In the absence of ATP, the tethered kinase is inactive. The soluble phosphatase enzymes dephosphorylate the peptide substrate. As a result, the antiphosphotyrosine antibody does not bind to the substrate, and the system produces a low Cy-5 fluorescent signal.

For each candidate drug, the soluble phosphatase enzymes are flushed out of the flow cell. A new buffer containing the candidate drug, and appropriate kinase cofactors (ATP, Magnesium, etc.) is flowed into the reaction cell. If the candidate drug does not inhibit the tethered kinase, the tethered kinase will phosphorylate the target peptide substrate, resulting in a high level of antiphosphotyrosine antibody binding, and hence a large Cy-5 fluorescent signal. However if the candidate drug inhibits the tethered kinase, the peptide will not be phosphorylated. Thus the Cy-5 fluorescent signal will remain small. Candidate drugs with intermediate degrees of kinase inhibition will produce intermediate levels of Cy-5 fluorescence increase.

The flow cell may be regenerated between each drug candidate test by flushing with another round of buffer containing soluble phosphatase enzymes, and low levels of ATP. Alternatively, a very low level of phosphatase enzyme may also be tethered to the hydrogel in addition to the other reaction components. This way, the microarray reagent will auto-regenerate (e.g. all of the peptide substrate will be gradually dephosphorylated) whenever the flow cell is flushed with buffer containing low levels of ATP.

By creating complex flow cells containing a variety of different bound enzymes, enzyme substrates, and (where appropriate, antibodies against a various state of an enzyme or enzyme substrate), the ability of drug candidates to specifically inhibit enzyme targets of interest may be rapidly assessed. Because these techniques require only trace amounts of candidate drugs and reagents, these methods offer some compelling advantages over the methods of prior art.

### EXAMPLE 5

Micro Fluidic Flow Cell Devices:

In many embodiments, it may be desirable to combine one or more test elements of the present invention into flow cells connected by micro-fluidic switch elements. Such micro-fluidic switches can be used to dynamically redirect the passage of a test ligand through a multiple of different detector elements, depending upon the results of earlier tests in the series. Usually, the micro-fluidic switches and detection elements will be computer controlled.

As an example, consider a multi-element device consisting of a series of potential target drug detector elements, a series of control (non drug-target) detector elements, a series of ADMET detector elements, and a high-performance, but low throughput, test ligand (drug candidate) analyzer such as a mass spectrometer. Here, micro fluidic switches may direct the passage of the test ligand through the system. As an example, depending upon the results of the drug target detector portion of the system, an inactive test ligand may be directed to a waste container, and subsequent non-target and/or ADMET analysis skipped. Those test ligands with appropriate activity may be directed to the ADMET section. In turn, those test ligands with appropriate ADMET characteristics may be directed to a general purpose, high-performance, test ligand analytical system, such as the mass spectrometer example given previously. This way, promising test ligands may be almost instantly identified and characterized.

Such micro fluidic-switched, multi-element methods, may also be desirable for speeding up test throughput. Typically, a set of unknown test ligands will only contain a few members with the desired activity. Since usually, no further analysis of test ligands without the desired binding or enzymatic inhibition capability is necessary, further analysis may be skipped and these test ligands may be discarded. Here, micro fluidic switching elements enable the creation of networked multi-element drug detection flow cell devices. Here, a large number of test ligands are screened in many different reaction cells for the desired reactivity. Those that are seen to have the appropriate activity are then directed by micro fluidic switches to a smaller number of control reaction cells, where a second stage of binding to non-target enzymes or receptors is assessed. Those few test ligands that pass this second stage test are then directed by micro fluidic switches to a relatively small number of ADMET flow cell sensors. At any point in the analysis, interesting test ligands can be diverted to a general-purpose high performance ligand analyzer, such as a mass spectrometer.

In this way, a high-performance micro-analytical drug discovery system may be created. Such a system could work using ultra small quantities of materials, such as the material created from a single combinatorial synthesis bead, and could significantly reduce the amount of materials, time, and effort required for the drug discovery process.

1: A reagent for measuring one or more test factors, said reagent comprising;
a ligand receptor protein, and a reagent ligand that can bind to a ligand binding site on said protein;
said protein and said reagent ligand being tightly coupled together by a tether that is chemically distinct from the protein and the reagent ligand;
wherein the test factors alter the ability of the reagent ligand to bind to the ligand binding site on said protein;
and wherein the binding or non binding of the reagent ligand to the ligand binding site on said protein creates a detectable signal.

2: The reagent of claim 1, in which the receptor protein is an antibody, and the reagent ligand contains an epitope recognized by the antibody.

3: The reagent of claim 1, in which the ligand receptor protein is an enzyme, and the reagent ligand is a substrate or inhibitor of the enzyme.

4: The reagent of claim 1, in which the reagent ligand binds to an allosteric site on the ligand receptor protein.

5: The reagent of claim 1, in which the test factor competes with the reagent ligand for binding to the ligand receptor on the ligand receptor protein.

6: The reagent of claim 1, in which the test factor modifies the conformation of the ligand receptor protein or the reagent ligand.

7: The reagent of claim 1, in which the test factor chemically modifies the ligand receptor protein or the reagent ligand.

8: The reagent of claim 1, in which the ligand receptor protein is labeled with a first moiety, and the reagent ligand is labeled with a second moiety;
in which either said first moiety or said second moiety emits photons; and the other moiety absorbs said photons by a resonance transfer mechanism;

9: The reagent of claim 1, in which the reagent is bound to a surface of a cellular component, bead, microarray, or flow cell.

10: A device for measuring one or more test factors,
said device containing a surface with one or more reagents tightly bound to said surface; said reagent or reagents comprising;
a ligand receptor protein, and a reagent ligand that can bind to a ligand binding site on said protein;
said protein and said reagent ligand being tightly coupled together by a tether that is chemically distinct from the protein and the reagent ligand;
wherein the test factors alter the ability of the reagent ligand to bind to the ligand binding site on said protein;
and wherein the binding or non binding of the reagent ligand to the ligand binding site on said protein alters the resonance transfer of photons between a first moiety associated with the protein, and a second moiety associated with the reagent ligand, producing a detectable signal;

11: The device of claim 10, in which the receptor protein is an antibody, and the reagent ligand contains an epitope recognized by the antibody.

12: The device of claim 10, in which the ligand receptor protein is an enzyme, and the reagent ligand is a substrate or inhibitor of the enzyme.

13: The device of claim 10, in which the reagent ligand binds to an allosteric site on the ligand receptor protein.

14: The device of claim 10, in which the test factor competes with the reagent ligand for binding to the ligand receptor on the ligand receptor protein.

15: The device of claim 10, in which the test factor modifies the conformation of the ligand receptor protein or the reagent ligand.

16: The device of claim 10, in which the test factor chemically modifies the ligand receptor protein or the reagent ligand.

17: The device of claim 10, in which the device is a component of a bead, microarray or flow cell.

18: A method of analyzing test factors, comprising:
applying said factors to a flow cell containing one or more reagents, said reagents comprising;
a ligand receptor protein, and a reagent ligand that can bind to a ligand binding site on said protein;
said protein and said reagent ligand being tightly coupled together by a tether that is chemically distinct from the protein and the reagent ligand;
wherein the ability of the test factors to cause changes in the detectable signal emitted by the reagents is observed.

19: The method of claim 18, in which multiple test factors are observed by the flow cell in a sequential manner

20: The method of claim 18, in which the flow cell is calibrated by one or more levels of control test factors.
